(19) United States
(12) Patent Application Publication
(10) Pub. No.: US 2023/0221388 A1
(43) Pub. Date: Jul. 13, 2023

(54) MAGNETIC RESONANCE IMAGING APPARATUS, IMAGE RECONSTRUCTION APPARATUS, AND IMAGE RECONSTRUCTION METHOD

(71) Applicant: CANON MEDICAL SYSTEMS CORPORATION, Tochigi (JP)

(72) Inventors: Fanxing MENG, Beijing (CN); Lijun ZHANG, Beijing (CN)

(73) Assignee: CANON MEDICAL SYSTEMS CORPORATION, Tochigi (JP)

(21) Appl. No.: 18/152,373

(22) Filed: Jan. 10, 2023

(30) Foreign Application Priority Data
Jan. 11, 2022 (CN) 202210025762.1
Jan. 6, 2023 (JP) 2023-001205

(51) Int. Cl. G01R 33/54; G01R 33/56

(52) U.S. Cl. CPC G01R 33/543; G01R 33/56

(57) ABSTRACT

A magnetic resonance imaging apparatus according to an aspect of the present disclosure includes a sequence controlling circuit and a processing circuit. The sequence controlling circuit is configured to acquire undersampled frequency domain scan data by executing a pulse sequence while carrying out an undersampling process. The processing circuit is configured: to generate image domain corrected data of the frequency domain scan data, by correcting the frequency domain scan data in an image domain; to generate frequency domain corrected data of the frequency domain scan data by correcting the frequency domain scan data in a frequency domain; to optimize the frequency domain scan data based on the image domain corrected data and the frequency domain corrected data; and to reconstruct image data by using the optimized frequency domain scan data.

### CROSS-REFERENCE TO RELATED APPLICATIONS

This application is based upon and claims the benefit of priority from Chinese Patent Application No. 202210025762.1, filed on Jan. 11, 2022; and Japanese Patent Application No. 2023-001205, filed on Jan. 6, 2023 the entire contents of all of which are incorporated herein by reference.

### FIELD

Embodiments described herein relate generally to a magnetic resonance imaging apparatus, an image reconstruction apparatus, and an image reconstruction method.

### BACKGROUND

In magnetic resonance imaging, a technique is known by which scan time of a magnetic resonance imaging apparatus is accelerated by acquiring only a part of frequency domain information so as to reconstruct an image from undersampled frequency domain data. This technique may be applied to a magnetic resonance imaging apparatus based on an algorithm of parallel imaging or compressed sensing. According to this technique, because the data of a part of the frequency domain information is missing, imaging quality may be degraded. In recent years, as machine learning techniques have been developed, magnetic resonance imaging apparatuses using deep learning are proposed. The machine learning techniques may be applied to correcting data of an image domain or a frequency domain or to structuring an end-to-end reconstruction neural network. By using the machine learning techniques, it is possible to increase the width of the scan acceleration and to improve imaging quality.

### BRIEF DESCRIPTION OF THE DRAWINGS

FIG. 1 is a diagram illustrating an example of a magnetic resonance imaging apparatus according to a first embodiment;

FIG. 2 is a diagram illustrating an example of a structure of a magnetic resonance image reconstruction apparatus according to the first embodiment;

FIG. 3 is a data flow chart for explaining a process performed by a K-space interpolating unit according to the first embodiment;

FIG. 4 is a data flow chart for explaining a process performed by a sensitivity distribution calculating unit according to the first embodiment;

FIG. 5 is a data flow chart for explaining a process performed by an image domain correcting unit according to the first embodiment;

FIG. 6 is a data flow chart for explaining a process performed by a consistency data calculating unit according to the first embodiment;

FIG. 7 is a data flow chart for explaining a process performed by a frequency domain correcting unit according to the first embodiment;

FIG. 8 is a data flow chart for explaining a process performed by an optimizing unit according to the first embodiment;

FIG. 9 is a data flow chart for explaining a process performed by a reconstructing unit according to the first embodiment;

FIG. 10 is a flowchart illustrating a flow in a magnetic resonance image reconstruction method according to the first embodiment;

FIG. 11 is a data flow chart for explaining steps S105 and S106 in the magnetic resonance image reconstruction method according to the first embodiment;

FIG. 12 is a diagram illustrating an example of a structure of a magnetic resonance image reconstruction apparatus according to a second embodiment; and

FIG. 13 is a flowchart illustrating a flow in a magnetic resonance image reconstruction method according to the second embodiment.

### DETAILED DESCRIPTION

A magnetic resonance imaging apparatus according to an aspect of the present disclosure includes a sequence controlling circuit and a processing circuit. The sequence controlling circuit is configured to acquire undersampled frequency domain scan data by executing a pulse sequence while carrying out an undersampling process. The processing circuit is configured: to generate image domain corrected data of the frequency domain scan data, by correcting the frequency domain scan data in an image domain; to generate frequency domain corrected data of the frequency domain scan data by correcting the frequency domain scan data in a frequency domain; to optimize the frequency domain scan data on the basis of the image domain corrected data and the frequency domain corrected data; and to reconstruct image data by using the optimized frequency domain scan data.

Exemplary embodiments of a magnetic resonance imaging apparatus, an image reconstruction apparatus, and an image reconstruction method will be explained in detail below, with reference to the accompanying drawings.

The following will describe the magnetic resonance imaging apparatus, the image reconstruction apparatus, and the image reconstruction method of the present disclosure, with reference to the accompanying drawings.

### First Embodiment

A magnetic resonance image reconstruction apparatus according to an embodiment of the present disclosure is configured to perform optimization n times in total (where n is an integer of 1 or larger) on frequency domain scan data K₀ in a K-space obtained from a scan performed on an examined subject (hereinafter, “patient”) by a magnetic resonance scanning apparatus and to obtain image data IMG on the basis of frequency domain scan data Kₙ resulting from the optimization performed n times. The magnetic resonance scanning apparatus is configured to obtain the frequency domain scan data K₀ of the patient, by transmitting a pulse signal to the patient placed in a magnetic field on which frequency encoding and phase encoding processes have been performed, and further receiving an echo signal caused by specific atomic magnetic resonance from a plurality of receiver coils.

In the present embodiment, the frequency domain scan data K₀ is a three-dimensional tensor expressed as a width W×a height H×the number of channels C (the number of coils), where the width direction corresponds to a frequency encoding direction, whereas the height direction corresponds to a phase encoding direction. Generally speaking, in magnetic resonance scans, undersampling is carried out by skipping specific frequency encoding for the purpose of shortening scan time. Thus, a scan is performed by skipping a part of the coordinates in the frequency encoding direction (the width direction) during the scan. For this reason, in the frequency domain scan data K₀, the part of the coordinates in the width direction (the frequency encoding direction) has no data, and a zero-padding process is performed for the data in the part of the coordinates. In the K-space, because the data in the vicinity of the center position has a larger impact on contrast of the reconstructed image data IMG, a method commonly used at the time of carrying out the undersampling is to sample, with priority, the data positioned in the vicinity of the center position in the frequency encoding direction, while skipping the data in some positions distant from the center position.

In the present embodiment, a mask M is used for indicating, during a magnetic resonance scan, which frequency encoding was sampled from the frequency domain scan data K₀. The mask M is a matrix of a width W×a height H, while the values of the coordinates in the frequency encoding and the phase encoding that were sampled are expressed as 1, whereas the values of the coordinates in the frequency encoding and the phase encoding that were skipped in the sampling are expressed as 0.

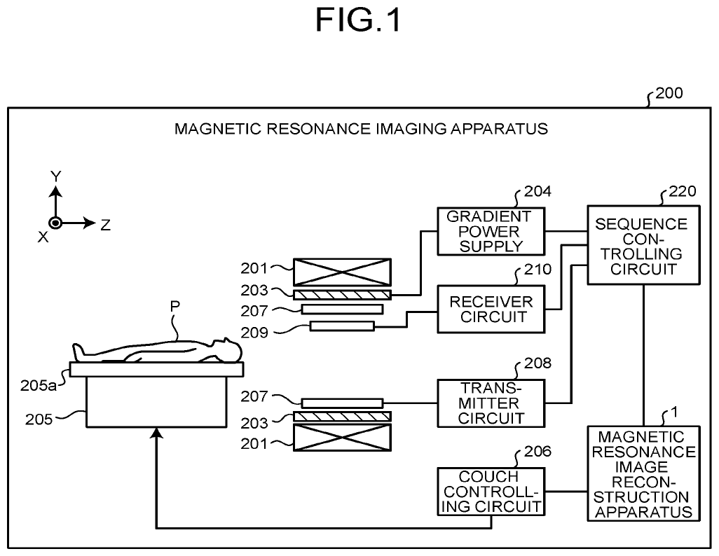

FIG. 1 is a block diagram illustrating a magnetic resonance imaging apparatus 200 according to the first embodiment. As illustrated in FIG. 1, the magnetic resonance imaging apparatus 200 includes a static magnetic field magnet 201, a static magnetic field power supply (not illustrated), a gradient coil 203, a gradient power supply 204, a couch 205, a couch controlling circuit 206, a transmitter coil 207, a transmitter circuit 208, a receiver coil 209, a receiver circuit 210, a sequence controlling circuit 220 (a sequence controlling unit), and a magnetic resonance image reconstruction apparatus 1. In this situation, a patient P (e.g., a human body) is not included in the magnetic resonance imaging apparatus 200. Further, the configuration illustrated in FIG. 1 is merely an example. For instance, it is acceptable to integrate or separate one or more of the functional units in the sequence controlling circuit 220 and the magnetic resonance image reconstruction apparatus 1, as appropriate.

The static magnetic field magnet 201 is a magnet formed so as to have a hollow substantially circular cylindrical shape and is configured to generate a static magnetic field in a space on the inside thereof. For example, the static magnetic field magnet 201 is a superconductive magnet or the like. In another example, the static magnetic field magnet 201 may be a permanent magnet.

The gradient coil 203 is a coil formed so as to have a hollow substantially circular cylindrical shape and is arranged on the inside of the static magnetic field magnet 201. The gradient coil 203 is formed by combining together three coils corresponding to X-, Y-, and Z-axes orthogonal to one another. By individually receiving a supply of an electric current from the gradient power supply 204, the three coils are configured to generate gradient magnetic fields of which magnetic field intensities change along the X-, Y-, and Z-axes. The gradient magnetic fields generated along the X-, Y-, and Z-axes by the gradient coil 203 are, for example, a slice gradient magnetic field Gs, a phase encoding gradient magnetic field Ge, and a readout gradient magnetic field Gr. The gradient power supply 204 is configured to supply the electric currents to the gradient coil 203.

The couch 205 includes a couchtop 205a on which the patient P is placed and is configured, under control of the couch controlling circuit 206, to insert the couchtop 205a into the hollow (an image taking opening) of the gradient coil 203, while the patient P is place thereon. Usually, the couch 205 is installed so that the longitudinal direction thereof is parallel to the central axis of the static magnetic field magnet 201. Under control of the magnetic resonance image reconstruction apparatus 1 (a computer), the couch controlling circuit 206 is configured to drive the couch 205, so as to move the couchtop 205a in longitudinal directions and up-and-down directions.

The transmitter coil 207 is arranged on the inside of the gradient coil 203 and is configured to generate a radio frequency magnetic field by receiving a supply of a Radio Frequency (RF) pulse from the transmitter circuit 208. The transmitter circuit 208 is configured to supply the RF pulse corresponding to a Larmor frequency determined by the type of targeted atoms and the magnetic field intensity, to the transmitter coil 207.

The receiver coil 209 is arranged on the inside of the gradient coil 203 and is configured to receive a magnetic resonance signal (hereinafter, “MR signal”, as necessary) emitted from the patient P due to influence of the radio frequency magnetic field. Upon receipt of the magnetic resonance signal, the receiver coil 209 is configured to output the received magnetic resonance signal to the receiver circuit 210.

The transmitter coil 207 and the receiver coil 209 described above are merely examples. It is possible to select one or combine two or more from among: a coil having only the transmitting function; a coil having only the receiving function; and a coil having the transmitting and receiving functions.

The receiver circuit 210 is configured to detect the magnetic resonance signal output from the receiver coil 209 and to generate magnetic resonance data on the basis of the detected magnetic resonance signal. More specifically, the receiver circuit 210 is configured to generate the magnetic resonance data by digitally converting the magnetic resonance signal output from the receiver coil 209. Further, the receiver circuit 210 is configured to transmit the generated magnetic resonance data to the sequence controlling circuit 220. Alternatively, the receiver circuit 210 may be provided for a gantry apparatus which includes the static magnetic field magnet 201, the gradient coil 203, and the like. Further, it is also acceptable to provide the receiver coil 209 with a part of the functions of the receiver circuit 210 such as, for example, the function to digitally convert the magnetic resonance signal.

The sequence controlling circuit 220 is configured to image the patient P, by driving the gradient power supply 204, the transmitter circuit 208, and the receiver circuit 210, on the basis of sequence information transmitted from the magnetic resonance image reconstruction apparatus 1 (the computer). In this situation, the sequence information is information defining a procedure for performing the imaging process. The sequence information defines: the magnitude of the electric current to be supplied to the gradient coil 203 by the gradient power supply 204 and the timing with which the electric current is to be supplied; the magnitude of the RF pulse to be supplied to the transmitter coil 207 by the transmitter circuit 208 and the timing with which the RF pulse is to be applied; the timing with which the magnetic resonance signal is to be detected by the receiver circuit 210; and the like. For example, the sequence controlling circuit 220 may be an integrated circuit such as an Application Specific Integrated Circuit (ASIC) or a Field Programmable Gate Array (FPGA), or an electronic circuit such as a Central Processing Unit (CPU) or a Micro Processing Unit (MPU). The sequence controlling circuit 220 is an example of a sequence controlling unit.

Further, upon receipt of the magnetic resonance data from the receiver circuit 210 as a result of imaging the patient P by driving the gradient power supply 204, the transmitter circuit 208, and the receiver circuit 210, the sequence controlling circuit 220 is configured to transfer the received magnetic resonance data to the magnetic resonance image reconstruction apparatus 1.

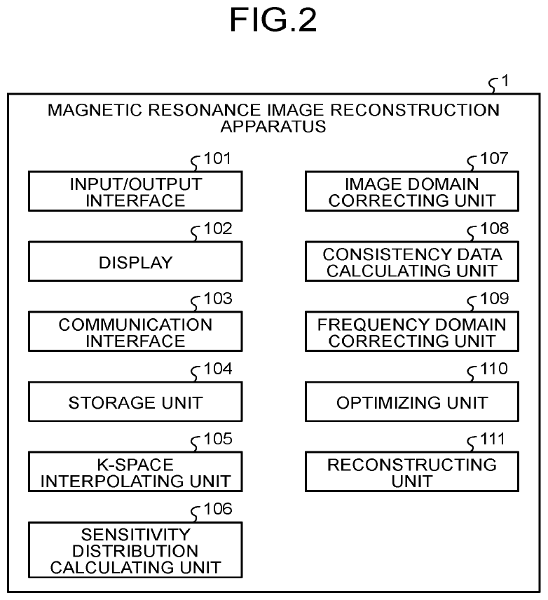

FIG. 2 is a diagram illustrating an example of a structure of the magnetic resonance image reconstruction apparatus 1 according to the first embodiment. The magnetic resonance image reconstruction apparatus 1 according to the first embodiment includes an input/output interface 101, a display 102, a communication interface 103, a storage unit 104, a K-space interpolating unit 105, a sensitivity distribution calculating unit 106, an image domain correcting unit 107, a consistency data calculating unit 108, a frequency domain correcting unit 109, an optimizing unit 110, and a reconstructing unit 111. The input/output interface 101, the display 102, the communication interface 103, the storage unit 104, the K-space interpolating unit 105, the sensitivity distribution calculating unit 106, the image domain correcting unit 107, the consistency data calculating unit 108, the frequency domain correcting unit 109, the optimizing unit 110, and the reconstructing unit 111 are connected so as to be able to communicate with one another.

The input/output interface 101 is an interface for connecting the magnetic resonance image reconstruction apparatus 1 to an input apparatus (not illustrated) and is configured to receive an input operation of a user from the input apparatus and to transfer a signal based on the received input operation to the magnetic resonance image reconstruction apparatus 1. The input/output interface 101 may be a serial bus interface such as a Universal Serial Bus (USB), for example. Examples of the input apparatus include a mouse, a keyboard, a trackball, a switch, a button, a joystick, a touch screen, and a microphone. Further, the input/output interface 101 may have a storage apparatus connected thereto, so as to be able to read and write various types of data from and to the storage apparatus. The storage apparatus may be a Hard Disc Drive (HDD), a Solid State Drive (SSD), or the like.

The display 102 is an interface for connecting the magnetic resonance image reconstruction apparatus 1 to a display apparatus (not illustrated) and is configured to transmit data to the display apparatus and to cause the display apparatus to display an image. For example, the display 102 is a picture output interface such as a Digital Visual Interface (DIV) or a High-Definition Multimedia Interface (HDMI (registered trademark)). Examples of the display apparatus include a Liquid Crystal Display (LCD) and an organic Electroluminescence (EL) Display. The display apparatus is configured to display a user interface for receiving input operations from the user and the image data IMG output by the magnetic resonance image reconstruction apparatus 1. Examples of the user interface include a Graphical User Interface (GUI).

The communication interface 103 is an interface for connecting the magnetic resonance image reconstruction apparatus 1 to a server (not illustrated) and is configured to be able to transmit and receive various types of data to and from the server. The communication interface 103 is, for example, a network card such as a wireless network card or a wired network card.

The storage unit 104 is configured to store therein the frequency domain scan data K₀ for performing image reconstruction and the mask M kept in correspondence therewith. Further, the storage unit 104 is configured to store therein parameters used by the magnetic resonance image reconstruction apparatus 1 at the time of performing the image reconstruction, such as parameters of a neural network, for example. Also, the storage unit 104 is configured store therein neural networks used by the magnetic resonance image reconstruction apparatus 1 and training data used for training other parameters that can be trained through machine learning. Sets of training data include the frequency domain scan data K₀, the mask M, and image data ground truth IMG_{GT}. For example, the storage unit 104 is realized by using a storage apparatus such as a Read Only Memory (ROM), a flash memory, a Random Access Memory (RAM), a Hard Disc Drive (HDD), a Solid State Drive (SSD), or a register. The flash memory, the HDD, the SSD, and the like are non-volatile storage media. These non-volatile storage media are realized by other storage apparatuses connected via a network such as a Network Attached Storage (NAS) or an external storage server apparatus. In this situation, examples of the network include the Internet, a Wide Area Network (WAN), a Local Area Network (LAN), a carrier terminal, a wireless communication network, a wireless base station, and a dedicated communication line.

A processing circuit (not illustrated) included in the magnetic resonance image reconstruction apparatus 1 has processing functions such as a K-space interpolating function, a sensitivity distribution calculating function, an image domain correcting function, a consistency data calculating function, a frequency domain correcting function, an optimizing function, and a reconstructing function. The processing functions implemented by the K-space interpolating function, the sensitivity distribution calculating function, the image domain correcting function, the consistency data calculating function, the frequency domain correcting function, and the optimizing function are stored in the storage unit 104 in the form of computer-executable programs. The processing circuit is a processor configured to realize the functions corresponding to the programs, by reading and executing the programs from the storage unit 104. In other words, the processing circuit that has read the programs has the abovementioned functions. That is to say, the K-space interpolating function, the sensitivity distribution calculating function, the image domain correcting function, the consistency data calculating function, the frequency domain correcting function, and the optimizing function, and the reconstructing function are examples of the K-space interpolating unit 105, the sensitivity distribution calculating unit 106, the image domain correcting unit 107, the consistency data calculating unit 108, the frequency domain correcting unit 109, the optimizing unit 110 and the reconstruction unit 111, respectively. Further, although FIG. 1 illustrates the example in which the single processing circuit realizes these processing functions, it is also acceptable to structure the processing circuit by combining together a plurality of independent processors so that the functions are realized as a result of the processors executing the programs. In other words, each of the abovementioned functions may be structured as a program, so that the single processing circuit executes the programs. In another example, a specific function may be installed in a dedicated independent program executing circuit.

The term “processor” used in the above description denotes, for example, a Central Processing Unit (CPU), a Graphical Processing Unit (GPU), or a circuit such as an Application Specific Integrated Circuit (ASIC) or a programmable logic device (e.g., a Simple Programmable Logic Device (SPLD), a Complex Programmable Logic Device (CPLD), or a Field Programmable Gate Array (FPGA)). The one or more processors are configured to realize the functions by reading and executing the programs saved in the memory 132.

Further, instead of having the programs saved in the storage unit 104, it is also acceptable to directly incorporate the programs in the circuits of the one or more processors. In that situation, the one or more processors realize the functions by reading and executing the programs incorporated in the circuits thereof.

On the basis of the mask M, the K-space interpolating unit 105 is configured to generate frequency domain interpolated data KSI by performing an interpolating process on a zero-padded part of the frequency domain scan data K₀. The K-space interpolating unit 105 includes a first neural network 1051 and a computing unit 1052. The first neural network 1051 may be, for example, a feed forward neural network, a convolutional neural network, or a transformer. Preferably, the first neural network 1051 is a convolutional neural network. More preferably, the first neural network 1051 is U-Net. In the present embodiment, the first neural network 1051 is assumed to be a convolutional neural network including an input layer, an output layer, a convolution layer, an activation layer, a pooling layer, a batch normalization layer, and a fully connected layer, while the sizes of the input layer and the output layer are equal to each other. Parameters used by the first neural network 1051 are stored in the storage unit 104. The computing unit 1052 is configured to convert the mask M into an inversed mask IM. The inversed mask IM is a matrix having the same size as that of the mask M and is obtained by replacing is in the mask M with 0s and replacing 0s in the mask M with 1s.

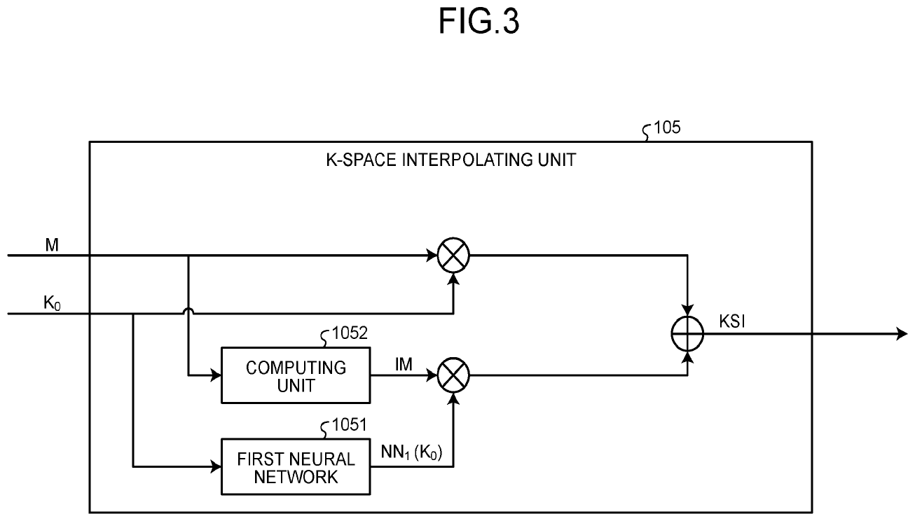

FIG. 3 is a data flow chart for explaining a process performed by the K-space interpolating unit 105 according to the first embodiment. In the following sections, the process performed by the K-space interpolating unit 105 will be explained with reference to FIG. 3. To begin with, the K-space interpolating unit 105 is configured to read the frequency domain scan data K₀ from the storage unit 104 and to input the frequency domain scan data K₀ to the first neural network 1051. The first neural network 1051 is configured to generate neural network data NN₁(K₀) having the same size as the frequency domain scan data K₀, by processing the frequency domain scan data K₀ by employing a trained neural network.

The process performed by the first neural network 1051 is considered as a process of embedding data in the zero-padded part of the frequency domain scan data K₀ on the basis of the data in the part of the frequency domain scan data K₀ that is not zero-padded. When the neural network data NN₁ (K₀) is compared with the frequency domain scan data K₀, the data has been embedded in the zero-padded part of the frequency domain scan data K₀, and the data in the part that is not zero-padded has also changed. Further, the K-space interpolating unit 105 is configured to read the mask M from the storage unit 104 and to cause the computing unit 1052 to calculate the inversed mask IM. After that, the K-space interpolating unit 105 is configured to calculate the frequency domain interpolated data KSI, by adding together the inner product of the neural network data NN₁(K₀) and the inversed mask IM and the inner product of the frequency domain scan data K₀ and the mask M.

When the frequency domain interpolated data KSI is compared with the frequency domain scan data K₀, only the zero-padded part of the frequency domain scan data K₀ is different, and the original data from the frequency domain scan data K₀ is retained in the other part. The frequency domain interpolated data KSI is considered as normally-sampled frequency domain scan data that was estimated by the K-space interpolating unit 105 on the basis of the undersampled frequency domain scan data K₀.

The sensitivity distribution calculating unit 106 is configured to generate a sensitivity distribution map SM by estimating sensitivities of a plurality of coils used by a magnetic resonance apparatus, on the basis of the frequency domain scan data K₀. The sensitivity distribution calculating unit 106 includes an inverse Fourier transform unit 1061, a second neural network 1062, and a computing unit 1063. The second neural network 1062 may be, for example, a feed forward neural network, a convolutional neural network, or a transformer. Preferably, the second neural network 1062 is a convolutional neural network. More preferably, the second neural network 1062 is U-net. In the present embodiment, the second neural network 1062 is assumed to be a convolutional neural network including an input layer, an output layer, a convolution layer, an activation layer, a pooling layer, a batch normalization layer, and a fully connected layer, while the sizes of the input layer and the output layer are equal to each other. Parameters used by the second neural network 1062 are stored in the storage unit 104. The computing unit 1063 is configured to convert neural network data NN₂(X₀) output by the second neural network 1062 into the sensitivity distribution map SM.

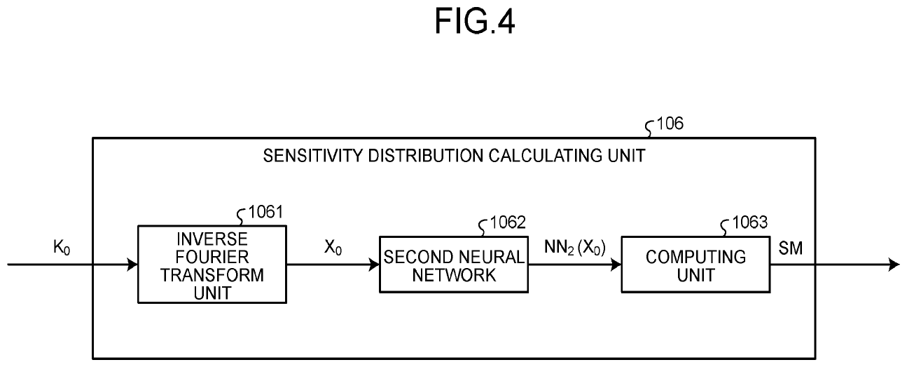

FIG. 4 is a data flow chart for explaining a process performed by the sensitivity distribution calculating unit 106 according to the first embodiment. In the following sections, the process performed by the sensitivity distribution calculating unit 106 will be explained, with reference to FIG. 4. To begin with, the sensitivity distribution calculating unit 106 is configured to generate image domain data X₀, by reading the frequency domain scan data K₀ from the storage unit 104 and causing the inverse Fourier transform unit 1061 to perform an inverse Fourier transform on the frequency domain scan data K₀. After that, the sensitivity distribution calculating unit 106 is configured to input the image domain data X₀ to the second neural network 1062. The second neural network 1062 is configured to generate the neural network data NN₂(X₀) having the same size as the image domain data X₀, by processing the image domain data X₀ by employing a trained neural network. The process performed by the second neural network 1062 is considered as processes of eliminating artifacts and eliminating noise from the image domain data X₀. After that, the sensitivity distribution calculating unit 106 causes the computing unit 1063 to calculate the sensitivity distribution map SM on the basis of the neural network data NN₂(X₀). More specifically, with respect to each of the voxels v included in the neural network data NN₂(X₀), voxels (including the voxel v itself) corresponding to the number of coils C having the same coordinate w in the width direction and the same coordinate h in the height direction with the voxel v are identified. The root sum square of the voxel values of the identified voxels is calculated, so as to subsequently calculate the quotient by dividing the voxel value of the voxel v by the root sum square and to determine the value of the quotient as the pixel value of the voxel v. The sensitivity distribution map SM is a three-dimensional tensor expressed as the width W×the height H×the number of coils C (the number of channels) and indicates the sensitivities of the coils. In the sensitivity distribution map SM, each of the matrices of the width W×the height H corresponding to a different one of the channels expresses the sensitivity of a corresponding one of the coils.

The image domain correcting unit 107 is configured to generate image domain corrected data IRₜ by correcting frequency domain scan data Kₜ that is obtained as a result of the optimizing unit 110 optimizing the frequency domain scan data K₀ t times, where t is an integer from 0 to n inclusive. The optimizing unit 110 and the optimizing process performed thereby will be explained later. The image domain correcting unit 107 includes an inverse Fourier transform unit 1071, a coil integrating unit 1072, a third neural network 1073, a coil separating unit 1074, and a Fourier transform unit 1075. The coil integrating unit 1072 is configured to convert multi-channel data into single-channel data. The third neural network 1073 may be, for example, a feed forward neural network, a convolutional neural network, or a transformer. Preferably, the third neural network 1073 is a convolutional neural network. More preferably, the third neural network 1073 is U-Net. In the present embodiment, the third neural network 1073 is assumed to be a convolutional neural network including an input layer, an output layer, a convolution layer, an activation layer, a pooling layer, a batch normalization layer, and a fully connected layer, while the sizes of the input layer and the output layer are equal to each other. Parameters used by the third neural network 1073 are stored in the storage unit 104. The coil separating unit 1074 is configured to convert single-channel data into multi-channel data.

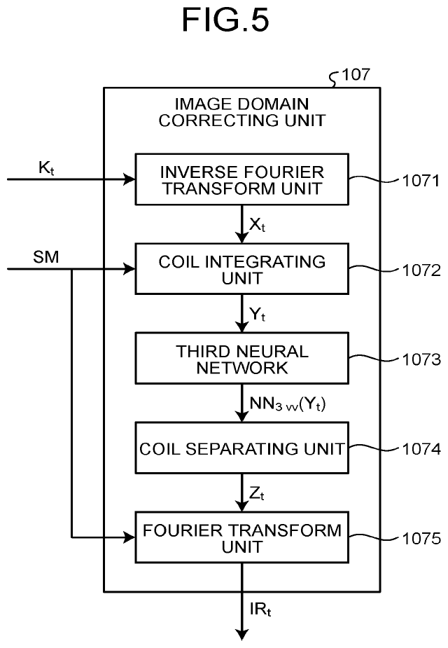

FIG. 5 is a data flow chart for explaining a process performed by the image domain correcting unit 107 according to the first embodiment. In the following sections, the process performed by the image domain correcting unit 107 will be explained, with reference to FIG. 5. To begin with, the image domain correcting unit 107 is configured to generate image domain data Xₜ, by reading the frequency domain scan data Kₜ from the storage unit 104 and causing the inverse Fourier transform unit 1071 to perform an inverse Fourier transform on the frequency domain scan data Kₜ. After that, the image domain correcting unit 107 is configured to cause the coil integrating unit 1072 to generate coil integrated data Yₜ, by integrating together the data of the plurality of coils in the image domain data Xₜ generated by the inverse Fourier transform unit 1071, on the basis of the sensitivity distribution map SM. The coil integrated data Yₜ is a matrix of the width W×the height H and is data obtained by integrating the channels corresponding to the number of coils C included in the image domain data Xₜ into a single channel, on the basis of the sensitivities of the coils indicated by the sensitivity distribution map SM. After that, the image domain correcting unit 107 is configured to input the coil integrated data Yₜ to the third neural network 1073. The third neural network 1073 is configured to generate neural network data NN₃(Yₜ) having the same size as the coil integrated data Yₜ by processing the coil integrated data Yₜ by employing a trained neural network. The process performed by the third neural network NN₃ is considered as processes of eliminating artifacts and eliminating noise from the coil integrated data Yₜ in an image domain. Further, the image domain correcting unit 107 is configured to generate coil separated data Zₜ by causing the coil separating unit 1074 to separate the single channel of the neural network data NN₃(Yₜ) into a plurality of channels, on the basis of the sensitivity distribution map SM. The coil separated data Zₜ is a three-dimensional tensor expressed as the width W×the height H×the number of coils C and is data obtained by separating the single channel in the neural network data NN₃(Yₜ) into the channels of the number of coils C corresponding to the coils, on the basis of the sensitivities of the coils. Further, the image domain correcting unit 107 is configured to generate image domain corrected data IRₜ, by causing the Fourier transform unit 1075 to perform a Fourier transform on the coil separated data Zₜ.

The consistency data calculating unit 108 is configured to generate consistency data DCₜ for constraining consistency between the frequency domain scan data Kₜ and the frequency domain scan data K₀.

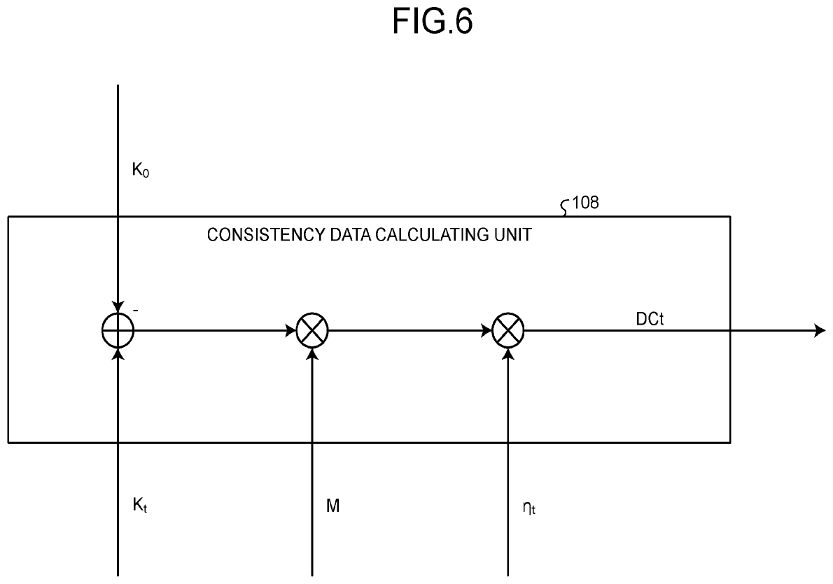

FIG. 6 is a data flow chart for explaining a process performed by the consistency data calculating unit 108 according to the first embodiment. In the following sections, the process performed by the consistency data calculating unit 108 will be explained, with reference to FIG. 6. To begin with, the consistency data calculating unit 108 is configured to read the frequency domain scan data K₀ and the frequency domain scan data Kₜ from the storage unit 104 and to calculate a difference tensor by subtracting the frequency domain scan data K₀ from the frequency domain scan data Kₜ. After that, the consistency data calculating unit 108 is configured to calculate the inner product of the mask M and the difference tensor. Further, the consistency data calculating unit 108 is configured to generate the consistency data DCₜ by multiplying the inner product by a learning parameter ηₜ. In this situation, the learning parameter ηᵢt is a parameter determined for each data set to be trained through machine learning. The learning parameter ηₜ has a value that is updated for each iteration. The initial value of the learning parameter is set to 1, for example.

The frequency domain correcting unit 109 is configured to generate frequency domain corrected data KRₜ, by correcting the frequency domain scan data Kₜ. The frequency domain correcting unit 109 includes a fourth neural network 1091. The fourth neural network 1091 may be, for example, a feed forward neural network, a convolutional neural network, or a transformer. Preferably, the fourth neural network 1091 is a convolutional neural network. More preferably, the fourth neural network 1091 is U-Net. In the present embodiment, the fourth neural network 1091 is assumed to be a convolutional neural network including an input layer, an output layer, a convolution layer, an activation layer, a pooling layer, a batch normalization layer, and a fully connected layer, while the sizes of the input layer and the output layer are equal to each other. Parameters used by the fourth neural network 1091 are stored in the storage unit 104.

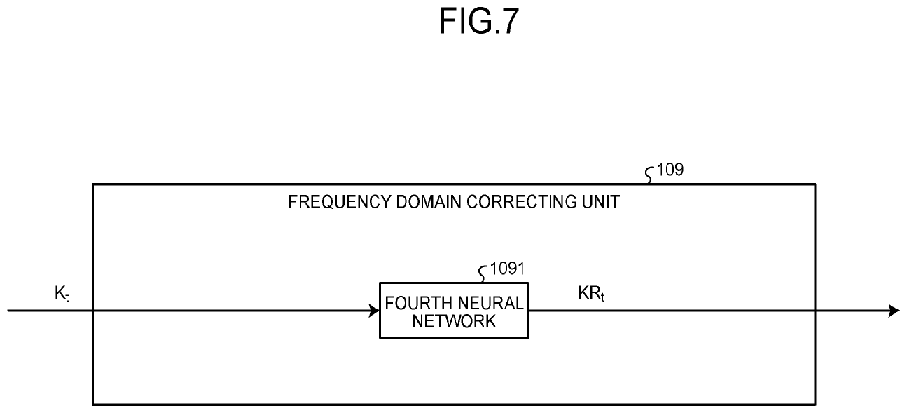

FIG. 7 is a data flow chart for explaining a process performed by the frequency domain correcting unit 109 according to the first embodiment. In the following sections, the process performed by the frequency domain correcting unit 109 will be explained, with reference to FIG. 7. The frequency domain correcting unit 109 is configured to read the frequency domain scan data Kₜ from the storage unit 104 and to input the frequency domain scan data Kₜ to the fourth neural network 1091. The fourth neural network 1091 is configured to generate frequency domain corrected data KRₜ having the same size as the frequency domain scan data Kₜ, by processing the frequency domain scan data Kₜ by employing a trained neural network. The process performed by the fourth neural network 1091 is considered as processes of eliminating artifacts and eliminating noise from the frequency domain scan data Kₜ in a frequency domain.

The optimizing unit 110 is configured to generate frequency domain scan data Kₜ₊₁, by optimizing the frequency domain scan data Kₜ, on the basis of the frequency domain interpolated data KSI, the image domain corrected data IRₜ, the consistency data DCₜ, and the frequency domain corrected data KRₜ.

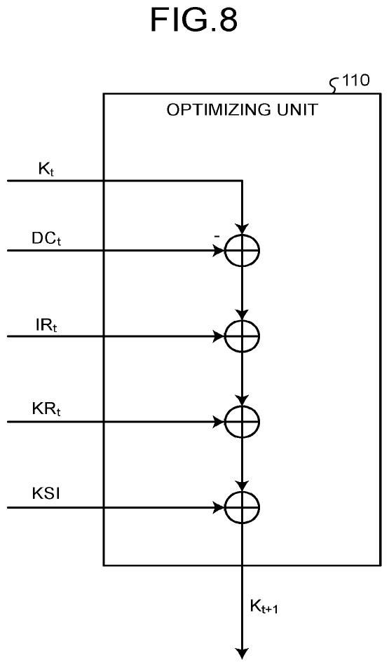

FIG. 8 is a data flow chart for explaining a process performed by the optimizing unit 110 according to the first embodiment. In the following sections, the process performed by the optimizing unit 110 will be explained with reference to FIG. 8. To begin with, the optimizing unit 110 is configured to calculate a difference tensor by subtracting the consistency data DCₜ from the frequency domain scan data Kₜ. After that, the optimizing unit 110 is configured to generate the frequency domain scan data Kₜ₊₁, by sequentially adding, to the difference tensor, the image domain corrected data IRₜ, the frequency domain corrected data KRₜ, and the frequency domain interpolated data KSI.

The reconstructing unit 111 is configured to generate the image data IMG, on the basis of the frequency domain scan data Kₙ resulting from performing optimization n times. The reconstructing unit 111 includes an inverse Fourier transform unit 1111 and a channel integrating unit 1112.

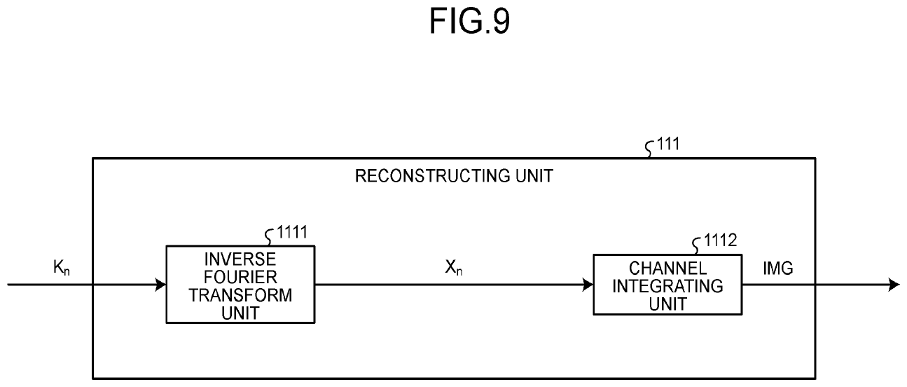

FIG. 9 is a data flow chart for explaining a process performed by the reconstructing unit 111 according to the first embodiment. In the following sections, the process performed by the reconstructing unit 111 will be explained, with reference to FIG. 9. Upon determination that the frequency domain scan data K₀ has been optimized n times, the reconstructing unit 111 is configured to read the frequency domain optimized data Kₙ from the storage unit 104 and to generate the image domain data Xₙ by causing the inverse Fourier transform unit 1111 to perform an inverse Fourier transform on the frequency domain optimized data Kₙ. After that, the reconstructing unit 111 is configured to cause the channel integrating unit 1112 to generate the image data IMG which is a matrix of the width W×the height H, on the basis of the image domain data Xₙ. More specifically, the channel integrating unit 1112 is configured to identify, with respect to each pixel p included in the image data IMG, pixels corresponding to the number of coils C having the same coordinate w in the width direction and the same coordinate h in the height direction with the pixel p from the image domain data Xₙ and to further determine the root sum square of the pixel values of these pixels to be the pixel value of the pixel p.

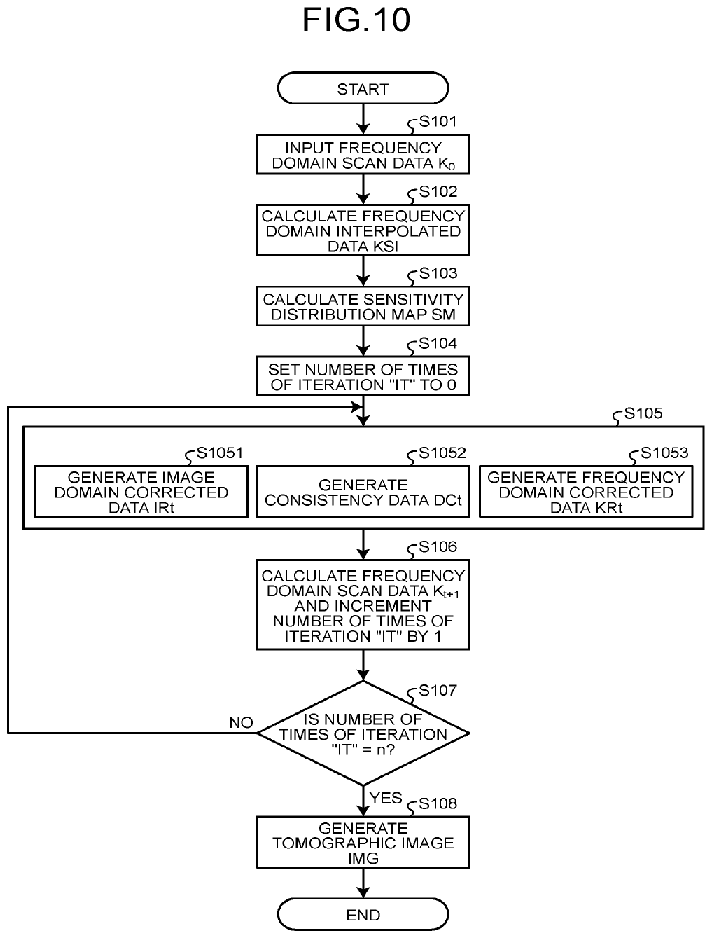

FIG. 10 is a flowchart illustrating a flow in a magnetic resonance image reconstruction method according to the first embodiment. In the following sections, the flow in the magnetic resonance image reconstruction method according to the first embodiment will be explained, with reference to FIG. 10.

The magnetic resonance image reconstruction method according to the present embodiment is configured to carry out the iteration n times in total, so that the undersampled frequency domain scan data K₀ gradually approaches normally-sampled frequency domain scan data KNS. In each iteration, the frequency domain scan data K₀ is optimized once. In the present embodiment, carrying out the iteration once denotes performing steps S105 and S106 once in the stated order. While the magnetic resonance image reconstruction method according to the present embodiment is implemented, the parameters of the neural networks and the other parameters that can be trained through machine learning are not changed. Preferably, the number of times of iteration is 6 to 12 times.

The sequence controlling circuit 220 is configured to acquire the undersampled frequency domain scan data K₀, by executing the pulse sequence while carrying out the undersampling process. At step S101, by using the user interface displayed on the display apparatus, the user selects, via the input apparatus, the frequency domain scan data K₀ and the mask M kept in correspondence with the frequency domain scan data K₀ that are either stored in the storage unit 104 or input from an external source. The process then proceeds to step S102.

At step S102, the K-space interpolating unit 105 generates the frequency domain interpolated data KSI by performing the interpolating process on the zero-padded part of the frequency domain scan data K₀ on the basis of the mask M. The process then proceeds to step S103. The frequency domain interpolated data KSI is used for optimizing the frequency domain scan data Kₜ in the iteration of n times thereafter.

At step S103, the sensitivity distribution calculating unit 106 generates the sensitivity distribution map SM by estimating the sensitivities of the plurality of coils used by the magnetic resonance apparatus, on the basis of the frequency domain scan data K₀. The process then proceeds to step S104. The sensitivity distribution map SM is used for calculating the frequency domain corrected data KRₜ in the iteration of n times thereafter. At step S104, the magnetic resonance image reconstruction apparatus 1 sets the number of times of iteration IT to 0. The process then proceeds to step S105.

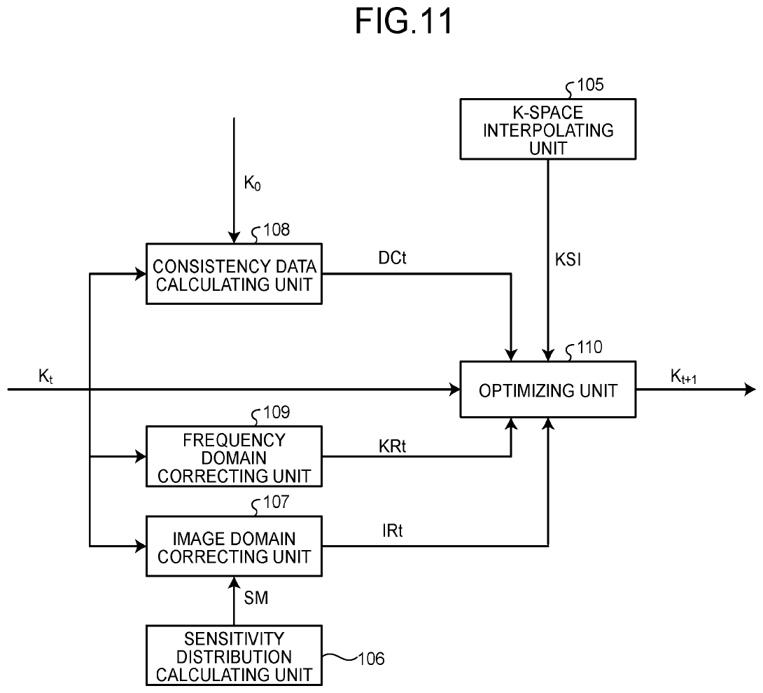

FIG. 11 is a data flow chart for explaining steps S105 and S106 in the magnetic resonance image reconstruction method according to the first embodiment. In the following sections, steps S105 and S106 will be explained, with reference to FIG. 11.

Step S105 includes steps S1051, S1052, and S1053. Steps S1051, S1052, and S1053 may be performed in parallel to one another or may be performed sequentially. In the present embodiment, the example in which steps S1051, S1052, and S1053 are performed in parallel to one another will be explained. At step S1051, the image domain correcting unit 107 generates the image domain corrected data IRₜ, by correcting the frequency domain scan data Kₜ on the basis of the sensitivity distribution map SM and outputs the image domain corrected data IRₜ to the optimizing unit 110. At step S1052, the consistency data calculating unit 108 generates the consistency data DCₜ for constraining consistency between the frequency domain scan data Kₜ and the frequency domain scan data K₀, on the basis of the frequency domain scan data Kₜ and the frequency domain scan data K₀ and further outputs the consistency data DCₜ to the optimizing unit 110. At step S1053, the frequency domain correcting unit 109 generates the frequency domain corrected data KRₜ by correcting the frequency domain scan data Kₜ and further outputs the frequency domain corrected data KRₜ to the optimizing unit 110. When all the processes at steps S1051, S1052, and S1053 are completed, the process proceeds to step S106.

Returning to the description of FIG. 10, at step S106, the optimizing unit 110 generates the frequency domain scan data Kₜ₊₁, by optimizing the frequency domain scan data Kₜ, on the basis of the frequency domain interpolated data KSI, the image domain corrected data IRₜ, the consistency data DCₜ, and the frequency domain corrected data KRₜ. The process then proceeds to step S107.

At step S107, the magnetic resonance image reconstruction apparatus 1 judges whether or not the number of times of iteration IT is equal to n. When the judgment result is “YES”, the process proceeds to step S108. When the judgment result is “NO”, the process proceeds to step S105. At step S108, the reconstructing unit 111 generates the image data IMG on the basis of the frequency domain optimized data Kₙ which is obtained by optimizing the frequency domain scan data K₀ n times, and the process is thus ended. At step S109, the magnetic resonance image reconstruction apparatus 1 increments the number of times of iteration IT by 1 and carries out the next iteration.

In the above explanation, the magnetic resonance image reconstruction apparatus and the magnetic resonance image reconstruction method according to the present embodiment are configured to use the first to the fourth neural networks and the parameter ηₜ. The neural networks and the parameter do not operate properly unless being trained. In the following sections, a method for training the abovementioned neural networks and parameter will be explained.

To begin with, a plurality of sets of training data that are stored in advance are read from the storage unit 104. The sets of training data include the undersampled frequency domain scan data K₀ obtained by the magnetic resonance apparatus and the mask M kept in correspondence therewith which serve as input data, as well as the image data ground truth IMG_{GT} which serves as output data.

Further, the plurality of sets of training data are divided into a training set and a test set. Examples of the ratio of the training set to the test set may include 80% to 20% and 90% to 10%. For example, when the total quantity of the sets of training data is 10,000, the training data numbered from #1 to #10,000 may be divided into the training set with the data numbered from #1 to #8,000; and the test set with the data numbered from #8,001 to #10,000. In this situation, the input data in the sets of training data within the training set is input to the magnetic resonance image reconstruction apparatus 1, further the image data IMG is calculated by implementing the magnetic resonance image reconstruction method according to the present embodiment, the difference value between the image data IMG and the image data ground truth IMG_{GT} is calculated, and a backpropagation is carried out on the basis of the difference value, so as to change the parameters of the neural networks and the other parameters that can be trained through the machine learning in such a manner that the difference value between the image data IMG output by the magnetic resonance image reconstruction apparatus 1 and the image data ground truth IMG_{GT} is reduced. The abovementioned process is repeatedly performed on a large part of the data in the training set until the difference value between the image data IMG output by the magnetic resonance image reconstruction apparatus 1 and the image data ground truth IMG_{GT} becomes smaller than a threshold value set in advance. After that, it is determined that the training of the neural networks and the parameters has been completed.

Subsequently, the test data (i.e., the data numbered from #8,000 to #10,000) serving as input data is input to the trained magnetic resonance image reconstruction apparatus 1 so as to calculate a difference value between the image data IMG output by the magnetic resonance image reconstruction apparatus 1 and the image data ground truth IMG_{GT} as evaluation data.

Next, advantageous effects of the magnetic resonance image reconstruction apparatus and the magnetic resonance image reconstruction method according to the present embodiment will be explained.

In a conventional magnetic resonance apparatus, to shorten the scan time, frequency domain information in a K-space is undersampled so as to generate undersampled frequency domain data. Because the reconstruction of a scan image using the undersampled frequency domain data is an ill-posed problem, there are countless solutions, and it is not possible to identify an accurate scan image. In contrast, the magnetic resonance image reconstruction apparatus and the magnetic resonance image reconstruction method according to the present embodiment are configured to solve the problem on the basis of a compressive sensing algorithm. The compression sensing algorithm is able to determine one appropriate solution in one-to-one correspondence, by adding a constraint.

The magnetic resonance image reconstruction method based on the compressive sensing is configured to calculate the normally-sampled frequency domain scan data KNS by iteratively optimizing the undersampled frequency domain scan data K₀. It is possible to express a relationship between the frequency domain scan data Kₜ₊₁ resulting from the optimization performed t+1 times and the frequency domain scan data Kₜ resulting from the optimization performed t times, by using expression (1) presented below.

Kₜ₊₁=Kₜ−ηₜ·DCₜ+G(Kₜ) (1)

In Expression (1), ηₜ denotes a learning parameter that can be trained through machine learning, whereas G(Kₜ) is a mathematical function of Kₜ. An additional constraint is expressed.

In the present embodiment, while the image domain corrected data IRₜ, the frequency domain corrected data KRₜ, and the frequency domain interpolated data KSI are used as the mathematical function G(Kₜ), a sparsity constraint is added to the image reconstruction.

The image domain corrected data IRₜ is corrected data obtained by correcting the frequency domain scan data Kₜ in the image domain, while employing the third neural network NN₃. The process of adding together the frequency domain scan data Kₜ and the image domain corrected data IRₜ is considered as processes of eliminating noise and eliminating artifacts from the frequency domain scan data Kₜ in the image domain, so as to cause the frequency domain scan data Kₜ to approach the normally-sampled frequency domain scan data KNS. The frequency domain corrected data KRₜ is corrected data obtained by correcting the frequency domain scan data Kₜ in the frequency domain, by employing a fourth neural network NN4. The process of adding together the frequency domain scan data Kₜ and the frequency domain corrected data KRₜ is considered as processes of eliminating noise and eliminating artifacts from the frequency domain scan data Kₜ in the frequency domain, so as to cause the frequency domain scan data Kₜ to approach the normally-sampled frequency domain scan data KNS. The frequency domain interpolated data KSI is data generated by the first neural network 1051 on the basis of the undersampled frequency domain scan data K₀ and is considered as normally-sampled frequency domain scan data estimated by the first neural network 1051. By adding the frequency domain interpolated data KSI multiplied by the learning parameter λₜ to the frequency domain scan data Kₜ, the frequency domain scan data Kₜ is caused to approach the normally-sampled frequency domain scan data KNS.

In an example of 4X undersampling, the image data IMG generated by the magnetic resonance image reconstruction apparatus 1 according to the present embodiment and the image data ground truth IMG_{GT} exhibit the following values: MSE=2.372e⁻¹¹±2.292e⁻¹¹; NMSE=0.003668±0.003615; PSNR=40.5±3.687; and SSIM=0.9514±0.3687. In an example of 8× undersampling, the image data IMG generated by the magnetic resonance image reconstruction apparatus 1 according to the present embodiment and the image data ground truth IMG_{GT} exhibit the following values: MSE=9.638e⁻¹¹±1.146e⁻¹⁰; NMSE=0.01261±0.00547; PSNR=34.77±4.609; and SSIM=0.9064±0.06189.

In the example of the 4X undersampling, the image data reconstructed by the magnetic resonance image reconstruction apparatus 1 according to the present embodiment restored the image data ground truth almost completely, and also, the processes of the noise elimination and the artifact elimination were performed on the image. In contrast, the image data reconstructed by using the conventional technique is unable to completely restore very small structures in the image, such as blood vessel. In addition, the image has apparent noise and artifacts. In the example of the 8X undersampling, the image data reconstructed by the magnetic resonance image reconstruction apparatus 1 according to the present embodiment roughly restored the image data ground truth, although a part of details of the image was lost. In addition, the processes of the noise elimination and the artifact elimination were performed on the image. In contrast, a large amount of detailed information was lost in the image data reconstructed by using the conventional technique. In addition, serious noise and artifacts occurred.

According to the present embodiment, because the neural networks are used for the K-space interpolating unit 105, the sensitivity distribution calculating unit 106, the image domain correcting unit 107, and the frequency domain correcting unit 109, it is possible to improve the precision level of the reconstruction for the image data IMG. In addition, it is also possible to reduce the number of times of iteration required before the image data IMG is reconstructed.

According to the present embodiment, the frequency domain scan data Kₜ is corrected by using the image domain corrected data IRₜ and the frequency domain corrected data KRₜ. It is therefore possible to reconstruct the image data IMG more accurately and in a more robust manner. Further, the image domain corrected data IRₜ and the frequency domain corrected data KRₜ are calculated in parallel to each other. It is therefore possible to enhance efficiency of the reconstruction and to improve the precision level of the reconstruction for the image data IMG.

According to the present embodiment, because the frequency domain scan data Kₜ is optimized by using the frequency domain interpolated data KSI, it is possible to reconstruct the image data IMG more accurately and in a more robust manner. Further, in the backpropagation during the training, it is possible to simplify the training process.

According to the present embodiment, because the image domain corrected data IRₜ is calculated by using the sensitivity distribution map SM, it is possible to further improve the precision level of the reconstruction for the image data IMG.

### Second Embodiment

Next, a magnetic resonance image reconstruction apparatus and a magnetic resonance image reconstruction method according to a second embodiment will be explained. In the second embodiment, differences from the first embodiment will primarily be explained. Explanations of some of the features that are the same as those in the first embodiment will be omitted. In the description of the second embodiment, some of the elements that are the same as those in the first embodiment will be referred to by using the same reference characters.

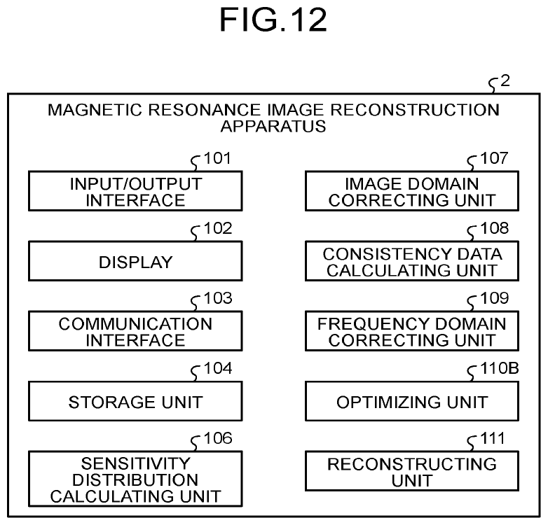

FIG. 12 is a diagram illustrating an example of a structure of a magnetic resonance image reconstruction apparatus 2 according to the second embodiment. In comparison to the first embodiment, the magnetic resonance image reconstruction apparatus 2 according to the second embodiment does not include the K-space interpolating unit 105, but includes an optimizing unit 110B in place of the optimizing unit 110. The rest of the structure is the same as that in the first embodiment.

The optimizing unit 110B is configured to generate frequency domain scan data Kₜ₊₁, by optimizing the frequency domain scan data Kₜ, on the basis of the image domain corrected data IRₜ, the consistency data DCₜ, and the frequency domain corrected data KRₜ.

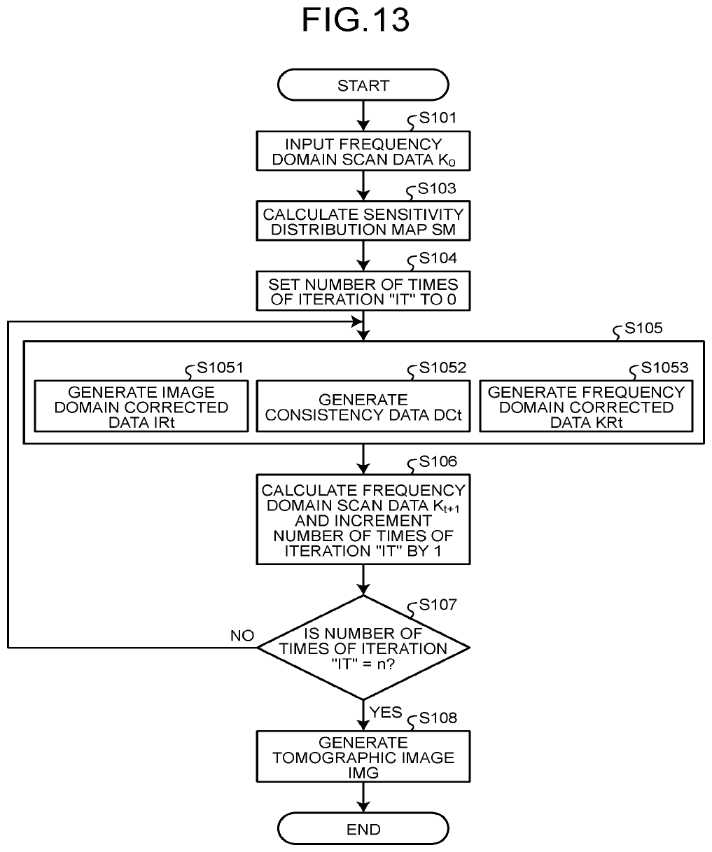

FIG. 13 is a flowchart illustrating a flow in the magnetic resonance image reconstruction method according to the second embodiment. In comparison to the first embodiment, the magnetic resonance image reconstruction method according to the second embodiment does not include step S102, while the other steps are the same as those in the first embodiment.

According to the present embodiment, because the frequency domain scan data Kₜ is corrected by using the image domain corrected data IRₜ and the frequency domain corrected data KRₜ, it is possible to reconstruct the image data IMG more accurately and in a more robust manner.

### Modification Examples

In the embodiments described above, the sensitivity distribution calculating unit 106 is configured to calculate the coil sensitivity distribution map SM on the basis of the neural network. However, the sensitivity distribution calculating unit 106 may be configured to calculate the coil sensitivity distribution map SM on the basis of an ESPiRiT algorithm. Further, it is also acceptable to reconstruct frequency domain scan data (a single channel) resulting from a scan performed by a single-coil magnetic resonance scanning apparatus. In addition, it is also acceptable to improve the precision level and resolution of the image data IMG by performing a super-resolution process on the frequency domain scan data K₀.

While a number of embodiments of the present disclosure have been described, these embodiments are presented by way of examples only, and are not intended to limit the scope of the inventions. The novel embodiments described herein may be carried out in a variety of other forms. It is also possible to make various omissions, substitutions, and changes without departing from the gist of the inventions. The embodiments and modifications thereof are covered by the scope and the gist of the inventions and are also covered by the present inventions and equivalents thereof. Further, it is also possible to carry out any of the embodiments described above in combination.

According to at least one aspect of the embodiments described above, it is possible to improve quality of the images.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A magnetic resonance imaging apparatus comprising:
a sequence controlling circuit configured to acquire undersampled frequency domain scan data by executing a pulse sequence while carrying out an undersampling process; and
a processing circuit configured
to generate image domain corrected data of the frequency domain scan data, by correcting the frequency domain scan data in an image domain,
to generate frequency domain corrected data of the frequency domain scan data by correcting the frequency domain scan data in a frequency domain,
to optimize the frequency domain scan data based on the image domain corrected data and the frequency domain corrected data, and
to reconstruct image data by using the optimized frequency domain scan data.

2. The magnetic resonance imaging apparatus according to claim 1, wherein
optimization is performed a predetermined number of times on the frequency domain scan data, and
the processing circuit is configured to optimize the optimized frequency domain scan data, based on the image domain corrected data of the optimized frequency domain scan data and the frequency domain corrected data of the optimized frequency domain scan data.

3. The magnetic resonance imaging apparatus according to claim 2, wherein
the processing circuit is configured to generate frequency domain interpolated data by performing an interpolating process on the frequency domain scan data that has not been optimized, and
the processing circuit is configured to optimize the optimized frequency domain scan data, based on the frequency domain interpolated data, the image domain corrected data of the optimized frequency domain scan data, and the frequency domain corrected data of the optimized frequency domain scan data.

4. The magnetic resonance imaging apparatus according to claim 3, wherein
the processing circuit is configured to calculate a sensitivity distribution map of a receiver coil, and
the processing circuit is configured to correct the frequency domain scan data based on the sensitivity distribution map.

5. The magnetic resonance imaging apparatus according to claim 4, wherein the processing circuit includes a neural network.

6. The magnetic resonance imaging apparatus according to claim 5, wherein the neural network is a convolutional neural network.

7. The magnetic resonance imaging apparatus according to claim 1, wherein
the processing circuit is configured to calculate consistency data for constraining consistency between the frequency domain scan data that has not been optimized and the optimized frequency domain scan data, and
the processing circuit is configured to optimize the frequency domain scan data, based on the consistency data, the image domain corrected data, and the frequency domain corrected data.

8. An image reconstruction apparatus comprising a processing circuit configured:
to correct undersampled frequency domain scan data in an image domain so as to generate image domain corrected data of the frequency domain scan data;
to generate frequency domain corrected data of the frequency domain scan data by correcting the frequency domain scan data in a frequency domain;
to optimize the frequency domain scan data based on the image domain corrected data and the frequency domain corrected data; and
to reconstruct image data by using the optimized frequency domain scan data.

9. The image reconstruction apparatus according to claim 8, wherein
optimization is performed a predetermined number of times on the frequency domain scan data, and
the processing circuit is configured to optimize the optimized frequency domain scan data, based on the image domain corrected data of the optimized frequency domain scan data and the frequency domain corrected data of the optimized frequency domain scan data.

10. The image reconstruction apparatus according to claim 9, wherein
the processing circuit is configured to generate frequency domain interpolated data by performing an interpolating process on the frequency domain scan data that has not been optimized, and
the processing circuit is configured to optimize the optimized frequency domain scan data, based on the frequency domain interpolated data, the image domain corrected data of the optimized frequency domain scan data, and the frequency domain corrected data of the optimized frequency domain scan data.

11. The image reconstruction apparatus according to claim 10, wherein
the processing circuit is configured to calculate a sensitivity distribution map of a receiver coil, and
the processing circuit is configured to correct the frequency domain scan data based on the sensitivity distribution map.

12. The image reconstruction apparatus according to claim 11, wherein the processing circuit includes a neural network.

13. The image reconstruction apparatus according to claim 12, wherein the neural network is a convolutional neural network.

14. The image reconstruction apparatus according to claim 8, wherein
the processing circuit is configured to calculate consistency data for constraining consistency between the frequency domain scan data that has not been optimized and the optimized frequency domain scan data, and
the processing circuit is configured to optimize the frequency domain scan data based on the consistency data, the image domain corrected data, and the frequency domain corrected data.

15. An image reconstruction method comprising:
correcting undersampled frequency domain scan data in an image domain so as to generate image domain corrected data of the frequency domain scan data;
generating frequency domain corrected data of the frequency domain scan data by correcting the frequency domain scan data in a frequency domain;
optimizing the frequency domain scan data based on the image domain corrected data and the frequency domain corrected data; and
reconstructing image data by using the optimized frequency domain scan data.

16. The image reconstruction method according to claim 15, wherein
optimization is performed a predetermined number of times on the frequency domain scan data, and
the optimized frequency domain scan data is optimized, based on the image domain corrected data of the optimized frequency domain scan data and the frequency domain corrected data of the optimized frequency domain scan data.

17. The image reconstruction method according to claim 16, wherein
frequency domain interpolated data is generated by performing an interpolating process on the frequency domain scan data that has not been optimized, and
the optimized frequency domain scan data is optimized, based on the frequency domain interpolated data, the image domain corrected data of the optimized frequency domain scan data, and the frequency domain corrected data of the optimized frequency domain scan data.

18. The image reconstruction method according to claim 17, wherein
a sensitivity distribution map of a receiver coil is calculated, and
the frequency domain scan data is corrected based on the sensitivity distribution map.

19. The image reconstruction method according to claim 18, wherein a neural network is used for correcting the frequency domain scan data on the basis of the sensitivity distribution map, generating the image domain corrected data, generating the frequency domain corrected data, and generating the frequency domain interpolated data.

20. The image reconstruction method according to claim 15, wherein
consistency data is calculated for constraining consistency between the frequency domain scan data that has not been optimized and the optimized frequency domain scan data, and
the frequency domain scan data is optimized based on the consistency data, the image domain corrected data, and the frequency domain corrected data.
